(19) United States
(12) Patent Application Publication
(10) Pub. No.: US 2024/0249799 A1
(43) Pub. Date: Jul. 25, 2024

(54) Systems and Methods for Cell Typing using GenoMaps

(71) Applicant: The Board of Trustees of the Leland Stanford Junior University, Stanford, CA (US)

(72) Inventors: Md Tauhidul Islam, Stanford, CA (US); Lei Xing, Palo Alto, CA (US)

(73) Assignee: The Board of Trustees of the Leland Stanford Junior University, Stanford, CA (US)

(21) Appl. No.: 18/411,996

(22) Filed: Jan. 12, 2024

Related U.S. Application Data
(60) Provisional application No. 63/479,724, filed on Jan. 12, 2023.

(51) Int. Cl. G16B 45/00; G06F 18/2415; G16B 40/00

(52) U.S. Cl. CPC G16B 45/00; G06F 18/2415; G16B 40/00

(57) ABSTRACT

Systems and methods for cell typing in accordance with embodiments of the invention are illustrated. One embodiment includes a cell typing system, including a processor, and a memory, the memory containing a cell typing application that configures the processor to obtain single cell ribonucleic acid sequencing (scRNA-seq) data generated from a single cell, generate a two-dimensional (2D) image includes a grid of pixels referred to as a GenoMap, where each pixel describes a gene-gene interaction based upon the scRNA-seq data, provide the 2D image to a convolutional neural network (CNN), obtain a cell classification of the single cell from the CNN, and provide the cell classification via a display.

### CROSS-REFERENCE TO RELATED APPLICATIONS

The current application claims the benefit of and priority under 35 U.S.C. § 119(e) to U.S. Provisional Patent Application No. 63/479,724 entitled “Cartography of Genomic Interactions Enables Deep Analysis of Single-Cell Expression Data” filed Jan. 12, 2023. The disclosure of U.S. Provisional Patent Application No. 63/479,724 is hereby incorporated by reference in its entirety for all purposes.

### FIELD OF THE INVENTION

The present invention generally relates to identifying cell types or cell states based on gene-gene interactions.

### BACKGROUND

RNA sequencing (RNA-seq) is a genomic technique for the detection and analysis of messenger RNA in a sample. In the past, RNA-seq was necessarily performed on samples containing many cells. Single-cell RNA-seq (scRNA-seq) has been enabled via advancements in laboratory technology and technique, and refers to the detection and analysis of messenger RNA from a single cell. Despite data derived from only a single cell, because of the large number of cells (thousands to millions) in a biological study, scRNA-seq data comes as a very large table (rows denoting cells and columns denoting genes).

Convolutional neural networks (CNNs) are a type of machine learning model characterized by at least one convolutional layer. CNNs have wide applications including image processing.

### SUMMARY OF THE INVENTION

Systems and methods for cell typing in accordance with embodiments of the invention are illustrated. One embodiment includes a cell typing system, including a processor, and a memory, the memory containing a cell typing application that configures the processor to obtain single cell ribonucleic acid sequencing (scRNA-seq) data generated from a single cell, generate a two-dimensional (2D) image includes a grid of pixels, where each pixel describes a gene-gene interaction based upon the scRNA-seq data, provide the 2D image to a convolutional neural network (CNN), obtain a cell classification of the single cell from the CNN, and provide the cell classification via a display.

In a further embodiment, to generate the 2D image, the cell typing application further directs the processor to generate a pairwise interaction strength matrix that maximizes entropy of the scRNA-seq data, generate a distance matrix for the 2D image, where the interaction strength matrix and the distance matrix have the same dimensions, optimize a transport function to produce a transport matrix coupling the distance matrix and the interaction strength matrix, and transpose the scRNA-seq data into the 2D image using the transport matrix.

In still another embodiment, to generate the pairwise interaction strength matrix, the cell typing application further directs the processor to maximize system entropy using a multivariate Gaussian distribution.

In a still further embodiment, the transport function is optimized using a Gromov-Wasserstein discrepancy.

In yet another embodiment, a loss function of the Gromov-Wasserstein discrepancy uses Kullback-Leibler divergence.

In a yet further embodiment, the Gromov-Wasserstein discrepancy is calculated using a regularized approximation.

In another additional embodiment, the CNN is trained using a training data set includes 2D images includes a grid of pixels, where each pixel describes a gene-gene interaction based upon a different scRNA-seq data, where each 2D image is labeled with a cell type from which the different scRNA-seq data was obtained.

In a further additional embodiment, the method further includes steps for a sequencer configured to generate the scRNA-seq data from a cell sample.

In another embodiment again, the cell typing application further directs the processor to provide the 2D image via the display.

In a further embodiment again, the CNN further provides a confidence metric reflecting probability of the cell classification being correct. 1Systems and methods for [PURPOSE] in accordance with embodiments of the invention are illustrated. One embodiment includes a method for cell typing, including obtaining single cell ribonucleic acid sequencing (scRNA-seq) data generated from a single cell, generating a two-dimensional (2D) image includes a grid of pixels, where each pixel describes a gene-gene interaction based upon the scRNA-seq data, providing the 2D image to a convolutional neural network (CNN), obtaining a cell classification of the single cell from the CNN, and providing the cell classification via a display. 12. The method for cell typing of claim 1, generating the 2D image includes generating a pairwise interaction strength matrix that maximizes entropy of the scRNA-seq data, generating a distance matrix for the 2D image, where the interaction strength matrix and the distance matrix have the same dimensions, optimizing a transport function to produce a transport matrix coupling the distance matrix and the interaction strength matrix, and transposing the scRNA-seq data into the 2D image using the transport matrix.

In still yet another embodiment, generating the pairwise interaction strength matrix includes maximizing system entropy using a multivariate Gaussian distribution.

In a still yet further embodiment, the transport function is optimized using a Gromov-Wasserstein discrepancy.

In still another additional embodiment, a loss function of the Gromov-Wasserstein discrepancy uses Kullback-Leibler divergence.

In a still further additional embodiment, the Gromov-Wasserstein discrepancy is calculated using a regularized approximation.

In still another embodiment again, the CNN is trained using a training data set includes 2D images includes a grid of pixels, where each pixel describes a gene-gene interaction based upon a different scRNA-seq data, where each 2D image is labeled with a cell type from which the different scRNA-seq data was obtained.

In a still further embodiment again, the method further includes steps for generating the scRNA-seq data from a cell sample using a sequencer.

In yet another additional embodiment, the method further includes steps for providing the 2D image via the display.

In a yet further additional embodiment, the CNN further provides a confidence metric reflecting probability of the cell classification being correct.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a cell typing system in accordance with an embodiment of the invention.

FIG. 2 illustrates a cell typing device in accordance with an embodiment of the invention.

FIG. 3 is a flow chart for a cell typing process in accordance with an embodiment of the invention.

FIG. 4 is a flow chart for a cell typing process for generating a GenoMap in accordance with an embodiment of the invention.

FIG. 5 illustrates example GenoMap for various cell types in accordance with an embodiment of the invention.

### DETAILED DESCRIPTION

Cell typing is a critical tool in both medicine and biological research. For example, cell typing can be used to investigate specific types of cancers and assist with directing disease treatments. However, conventional analytical techniques identifying individual cells from scRNA-seq data such as (but not limited to) discriminant analysis, Bayesian classification, decision-trees, and neural networks, all are deficient in extracting the most discriminative features. In part, this is because scRNA-seq data is typically stored as a vector or matrix, which is convenient from a data storage perspective but is not the most informative way to present the data. When stored in this manner, the information of gene-gene interactions is buried in the unordered expression matrix.

Systems and methods described herein describe and utilize GenoMap, a new structure for representing scRNA-seq data in a two-dimensional (2D) format which relates a gene placement configuration in 2D and a gene-gene interaction matrix computed from high dimension. In many embodiments, the gene-gene interaction matrix is computed by maximizing the entropy of the genomic data. As the possible combinations of placing the genes into a 2D grid is a factorial of the number of involved genes (typically around 20,000 for a human cell), a robust optimization can have significant impact on subsequent analysis. GenoMaps possess the basic characteristic of an image where the pixel configuration is determined by the gene-gene interactions of the cell. After GenoMap construction, a convolutional neural network can be used to extract genomic interaction features in order to type the cell. Turning now to the drawings, systems and methods for cell typing using GenoMaps are illustrated. Systems for cell typing are discussed first below.

### Cell Typing Systems

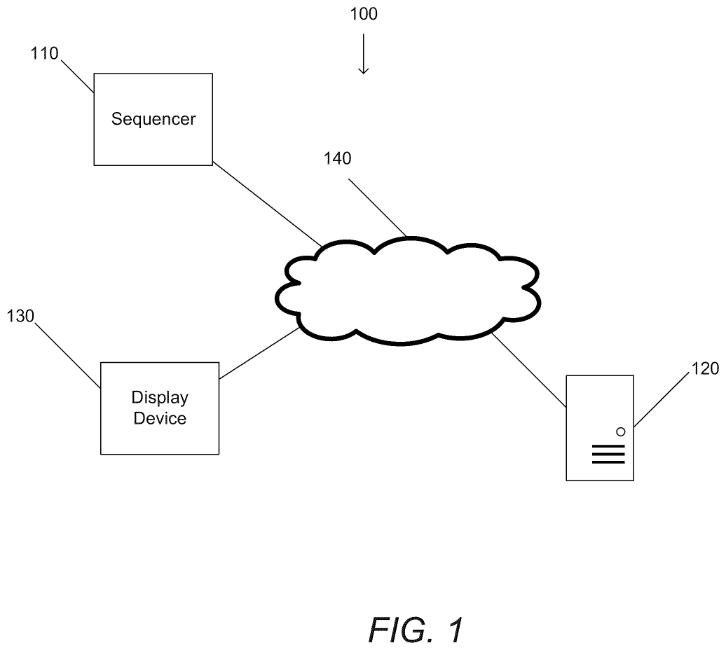

Cell typing systems are computational systems which obtain scRNA-seq data and convert said data into a GenoMap. In many embodiments, cell typing systems further utilize CNNs to process GenoMaps in order to type cells. Turning now to FIG. 1, a system architecture for a cell typing system in accordance with an embodiment of the invention is illustrated. System 100 includes a sequencer 110. Sequencers are devices which read sequences from cell samples and produce scRNA-seq data. The scRNA-seq data is transformed into a GenoMap by typing device 120. Typing devices are computer platforms which can produce GenoMaps and further process them in order to type cells. Results are displayed via a display device 130. The sequencer 110, typing device 120, and display device 130 are communicatively coupled by a network 140. In many embodiments, the network is the Internet, however any networking modality including wired networking, wireless network, or any combination of networks thereof can be used to connect one or more devices. In numerous embodiments, the display device and typing device are implemented using the same computing platform. Similarly, a sequencer may include the necessary computing hardware to act as a typing device and/or a display.

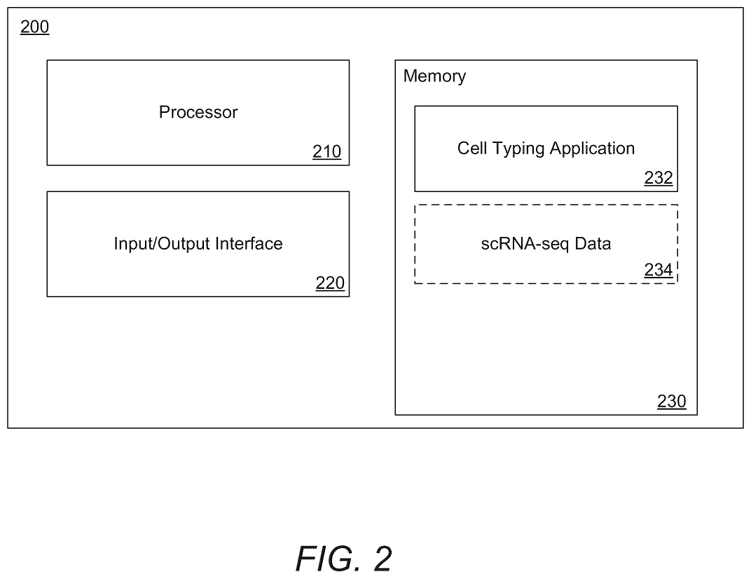

Turning now to FIG. 2, a block diagram for a typing device in accordance with an embodiment of the invention is illustrated. Typing device 200 includes a processor 210. Processors can be any logic circuitry capable of executing computation in accordance with cell typing processes. For example, processors can be implemented as a central processing unit (CPU), a graphics processing unit (GPU), an application-specific integrated circuit (ASIC), a field-programmable gate array (FPGA), and/or any combination thereof or alternative logic processing circuit. Typing device 200 further includes an input/output (I/O) interface 220 capable of communicating with connected devices, and a memory 230. Memory can be implemented using volatile memory, non-volatile memory, or any combination thereof. Memory 230 contains a cell typing application 232 which is capable of configuring the processor to execute cell typing processes as described herein. In numerous embodiments, the memory also contains scRNA-seq data generated by a sequencer.

While a specific system architecture and typing device architecture are illustrated in FIGS. 1 and 2, as can readily be appreciated, any number of different computing platforms can be used without departing from the scope or spirit of the invention including (but not limited to) cloud computing platforms, and/or any other computing platform with sufficient computational power to execute cell typing processes as described herein. Said cell typing processes are described in further detail below.

### Cell Typing Processes

Cell typing processes as described herein use GenoMaps as an intermediary between scRNA-seq data and a classification. In numerous embodiments, the type of cell may be readily apparently from the GenoMap alone to a human user. However, in various embodiments, machine learning is used to classify a cell using a GenoMap with significantly higher precision than possible by human eye alone.

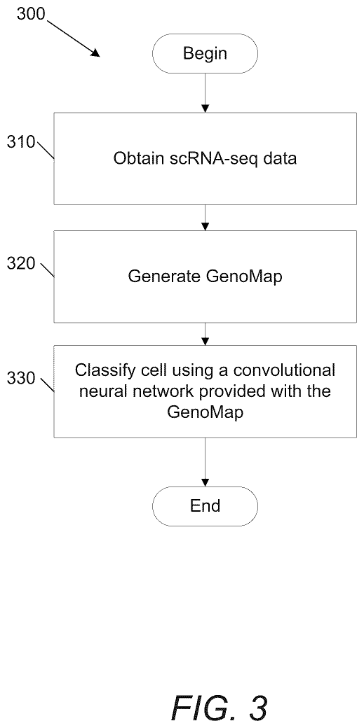

Turning now to FIG. 3, a high-level process for typing a cell in accordance with an embodiment of the invention is illustrated. Process 300 includes obtaining (310) scRNA-seq data for a given cell. A GenoMap is generated (320) from the scRNA-seq data, which is then provided to a CNN. The CNN then produces a classification (330) based on the GenoMap. In many embodiments, the CNN is previously trained using a training data set that contains GenoMaps derived from data labeled with known cell types. In many embodiments, the CNN further provides a confidence metric reflecting the probability of the predicted cell type being correct.

In order to reconfigure scRNA-seq data into a GenoMap, first, a pairwise interaction strength matrix that maximizes the entropy of the scRNA-seq data is generated. Then the genes in the scRNA-seq data are placed in a 2D grid such that the pairwise interaction is preserved maximally. An optimal transport optimization (i.e. minimization of Gromov-Wasserstein discrepancy between the interaction-space of genes and the Euclidean space of the 2D grid) can be used to solve the problem efficiently.

Assuming a data set Iₛ∈ [symbol] ^{m×n} from an experiment on m number of cells (each cell has n number of genes), the objective is to restructure the n genes of each cell into a 2D grid of size p×q, p×q≥n to maximize the entropy of the data. Entropy is frequently used in information theory to measure the information content of a system. Mathematically, entropy measures the uncertainty associated with a random variable or a random system. The entropy H(X) of a discrete random variable X can be written as:

$$H ( X ) = - {\sum }_{x ∈ 𝒳} p ( x ) log p ( x )$$

where p( [symbol] )=P(X= [symbol] ), [symbol] ∈ [symbol] , denotes the probability mass function (pmf) of the random variable X, and [symbol] is a finite set (such as {1,2, . . . }).

The scRNA-seq data for a cell of n genes can be written as a state vector [symbol] =( [symbol] ₁, . . . , [symbol] ₙ)), where [symbol] ᵢ is the expression level of the i-th gene. For m number of cells, there are m state vectors [symbol] ¹ . . . [symbol] ^{m}. The goal is to restructure the genes in each cell in such a way that maximizes the entropy of the gene expression vectors:

$$H = - {\sum }_{{x}^{→︀}} p ( \vec{x} ) ln p ( \vec{x} )$$

subject to the constraints

$${\sum }_{{χ}^{→︀}} p ( \vec{x} ) = 1 .$$

The probability mass function for the gene expressions, which maximizes the system entropy is given by a multivariate Gaussian distribution parametrized by the mean (x) and the covariance matrix Ω as follows:

$$P ( x ; 〈 x 〉 , Ω ) = {( 2 π )}^{- n / 2} {det ( Ω )}^{- 1 / 2} exp ( - \frac{1}{2} {( x - 〈 x 〉 )}^{T} {Ω}^{- 1} ( x - 〈 x 〉 ) ) .$$

Here, the covariance matrix is defined as:

$${Ω}_{i j} = ( {x}_{i} {x}_{j} 〉 - 〈 {x}_{i} 〉 ( {x}_{j} 〉 ,$$

$$Where$$

$$〈 {x}_{i} 〉 = {\sum }_{{χ}^{→︀}} p ( \vec{x} ) {x}_{i} = \frac{1}{m} {\sum }_{k = 1}^{m} {x}_{i}^{k} ,$$

$$And$$

$$( {x}_{i} {x}_{j} 〉 = {\sum }_{{χ}^{→︀}} p ( \vec{x} ) {x}_{i} {x}_{j} = \frac{1}{m} {\sum }_{k = 1}^{m} {x}_{i}^{k} {x}_{j}^{k} .$$

The pairwise interaction strength between [symbol] ᵢ and [symbol] ⱼ can then be computed from the covariance matrix as follows:

$${ρ}_{i j} = \{ \begin{matrix}- \frac{{( {Ω}^{- 1} )}_{i j}}{\sqrt{{( {Ω}^{- 1} )}_{i i} {( {Ω}^{- 1} )}_{j j}}} & if i \neq j , \\ 1 & if i = j .\end{matrix}$$

The problem of constructing the GenoMap (i.e. optimally placing n-genes to n positions of the 2D grid of p×q(n≤p×q)) can be written as Gromov-Wasserstein discrepancy between the scaled pair interaction strength matrix C of n genes and the distance matrix (C̅) of the 2D grid space. Both the matrices C and C̅ are of size n×n. The Gromov-Wasserstein discrepancy between matrices C and C̅ is defined as follows:

$$G W ( C , \overline{C} , u , v ) {=}^{def .} {min}_{T ∈ {C}_{u , ν}} {ε}_{C , \overline{C}} ( T ) ,$$

$$where$$

$${ε}_{C , \overline{C}} ( T ) {=}^{def .} {\sum }_{i , j , k , ℓ} L ( {C}_{i , k} , {\overline{C}}_{j , ℓ} ) {T}_{i , j} {T}_{k , ℓ} .$$

Here, the transport matrix T is a coupling between the two spaces on which C and C̅ are defined, u and v are vectors containing relative importance of the genes and the locations in the GenoMap. L here is a loss function to account for the discrepancy between the matrices and defined as the Kullback-Leibler divergence L(a, b)=KL(a|b) [symbol] a log(a/b)−a+b. Introducing the 4-way tensor:

$$L ( C , \overline{C} ) {=}^{def .} {( L ( {C}_{i , k} , {\overline{C}}_{j , ℓ} ) )}_{i , j , k , ℓ} ,$$

$$then$$

$${ℰ}_{c , \overline{c}} ( T ) = 〈 ℒ ( C , \overline{C} ) ⊗ T , T 〉 .$$

Here ⊗ denotes the tensor-matrix multiplication as follows:

$$ℒ ⊗ T {=}^{def .} {( {\sum }_{k , ℓ} {ℒ}_{i , j , k , ℓ} {T}_{k , ℓ} )}_{i , j} .$$

In many embodiments, a regularized Gromov-Wasserstein discrepancy is used for computational efficiency. The regularized approximation of the original Gromov-Wasserstein formulation is:

$$G {W}_{ε} ( C , \overline{C} , u , v ) {=}^{def .} {min}_{T ∈ {C}_{p , g}} {ε}_{C , \overline{C}} ( T ) - ε H ( T ) ,$$

where ε is a regularization parameter and the entropy of T∈ [symbol] ₊^{n×n} is defined as

$$H ( T ) {=}^{def .} - {\sum }_{i , j = 1}^{n} {T}_{i , j} ( log ( {T}_{i , j} ) - 1 ) .$$

A projected gradient descent is used to solve nonconvex optimization problem, where both the gradient step and the projection are computed according to the KL metric. Iterations of this algorithm are given by

$$T ← {Proj}_{{ε}_{p , q}}^{K L} ( T ⊙ {e}^{- τ ( ∇ {ε}_{c , c} ( T ) - ε ∇ H ( T ) )} ) ,$$

where τ>0 is a small step size, and the KL projector of any matrix K is:

$${Proj}_{{C}_{p , q}}^{K L} ( K ) {=}^{def .} {argmin}_{{T}^{′} ∈ {C}_{p , q}} KL ( {T}^{′} \vert K ) .$$

In the special case τ=1/ε, then

$$T ← 𝒯 ( ℒ ( C , \overline{C} ) ⊗ T , p , q ) .$$

In order to additionally speed up computation, if the loss can be written as

$$L ( a , b ) = {f}_{1} ( a ) + {f}_{2} ( b ) - {h}_{1} ( a ) {h}_{2} ( b )$$

for functions (f₁, f₂, h₁, h₂), then, for any T∈ [symbol] _{u,v}⁷⁸,

$$ℒ ( C , \overline{C} ) ⊗ T = {c}_{c , \overline{c}} - {h}_{1} ( C ) T {{h}_{2} ( \overline{C} )}^{T} ,$$

where c_{C,C̅} is independent of T. For this class of losses, [symbol] (C,C̅) ⊗T can be computed efficiently in O(n²n+n²n) operations, using only matrix/matrix multiplications, instead of the O(n²n²) complexity of a naïve implementation. In this case, the KL loss satisfies the above loss equation for f₁(a)=a log(a)−a, f₂(b)=b, h₁(a)=a, and h₂(b)=log(b).

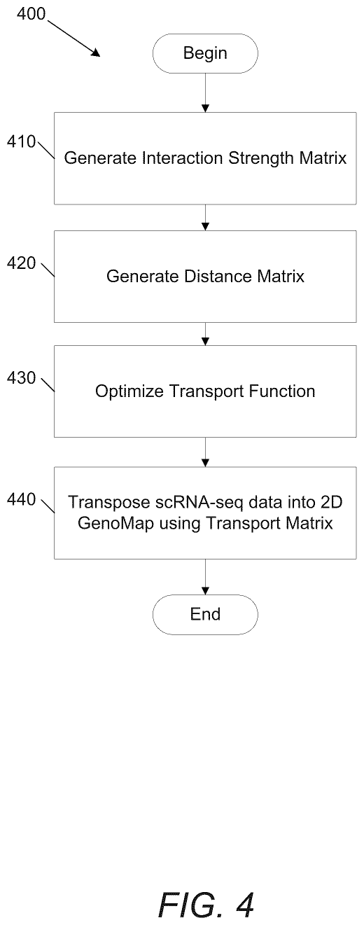

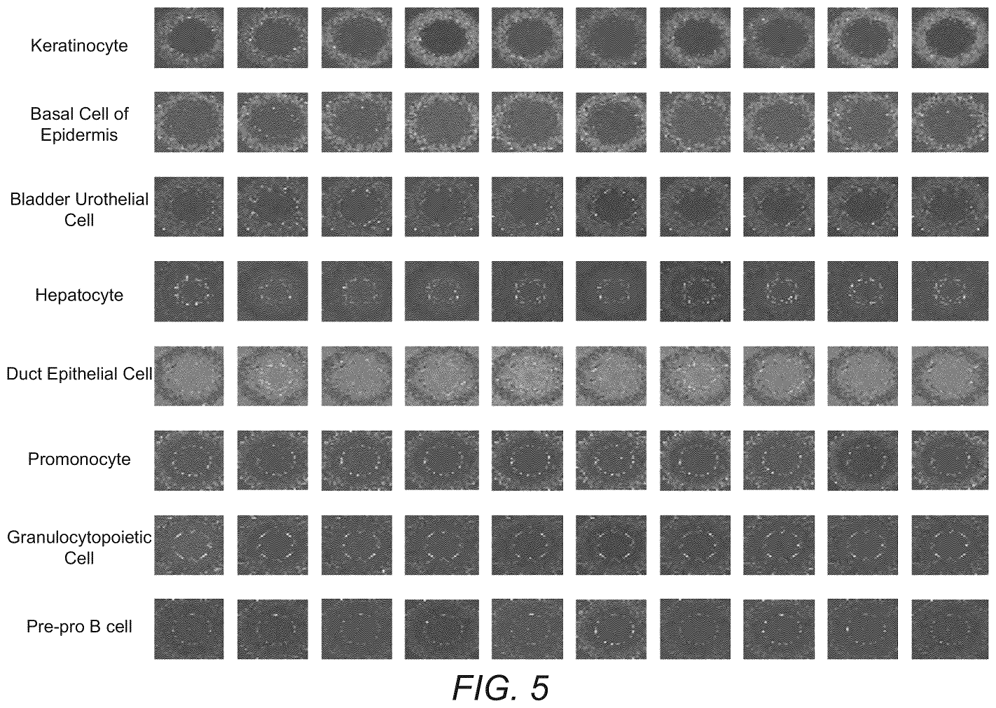

Turning now to FIG. 4, a flow chart for a GenoMap construction process in accordance with an embodiment of the invention is illustrated. Process 400 includes generating (410) the interaction strength matrix from the scRNA-seq data and generating (420) a distance matrix for a 2D space, where both the interaction strength matrix and distance matrix are n×n, where n is the number of genes in the scRNA-seq data. A transport function is optimized (430) between the two matrices resulting in a transport matrix. The transport matrix is used to transpose (440) the scRNA-seq data into the 2D space as a GenoMap. Example GenoMaps generated from different example cells for example cell types are illustrated in FIG. 5. As can be seen, each cell type produces a different and distinct GenoMap. While particular processes are illustrated with respect to FIGS. 3 and 4, different optimizations may be implemented without departing from the scope or spirit of the invention. As previously noted, the GenoMaps can be used for any number of different applications, including (but not limited to) cell typing.

GenoMaps can also be used for a wide variety of applications (but not limited to) including cell clustering, gene signature extraction, single cell data integration, and cellular trajectory analysis. Furthermore, while GenoMaps are discussed with respect to scRNA-seq data, a similar mathematical approach can be implemented with respect to any high-dimensional tabular data set to graphically represent them in the general form of a GenoMap, the TabMap.

In this way, complex datasets can be depicted in imaging format, with the relationships among the data components encoded in terms of the pixelated configuration. These configurations can then be processed using CNNs, which are highly effective tools for image processing. TabMaps in their general form can be used for any dataset specific task such as (but not limited to) dimensionality reduction, visualization, and/or any other function that a machine learning model can perform. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A cell typing system, comprising:
a processor; and
a memory, the memory containing a cell typing application that configures the processor to:
obtain single cell ribonucleic acid sequencing (scRNA-seq) data generated from a single cell;
generate a two-dimensional (2D) image comprising a grid of pixels, where each pixel describes a gene-gene interaction based upon the scRNA-seq data;
provide the 2D image to a convolutional neural network (CNN);
obtain a cell classification of the single cell from the CNN; and
provide the cell classification via a display.

2. The cell typing system of claim 1, wherein to generate the 2D image, the cell typing application further directs the processor to:
generate a pairwise interaction strength matrix that maximizes entropy of the scRNA-seq data;
generate a distance matrix for the 2D image, where the interaction strength matrix and the distance matrix have the same dimensions;
optimize a transport function to produce a transport matrix coupling the distance matrix and the interaction strength matrix; and
transpose the scRNA-seq data into the 2D image using the transport matrix.

3. The cell typing system of claim 2, wherein to generate the pairwise interaction strength matrix, the cell typing application further directs the processor to maximize system entropy using a multivariate Gaussian distribution.

4. The cell typing system of claim 3, wherein the transport function is optimized using a Gromov-Wasserstein discrepancy.

5. The cell typing system of claim 4, wherein a loss function of the Gromov-Wasserstein discrepancy uses Kullback-Leibler divergence.

6. The cell typing system of claim 4, wherein the Gromov-Wasserstein discrepancy is calculated using a regularized approximation.

7. The cell typing system of claim 1, wherein the CNN is trained using a training data set comprising 2D images comprising a grid of pixels, where each pixel describes a gene-gene interaction based upon a different scRNA-seq data, where each 2D image is labeled with a cell type from which the different scRNA-seq data was obtained.

8. The cell typing system of claim 1, further comprising a sequencer configured to generate the scRNA-seq data from a cell sample.

9. The cell typing system of claim 1, wherein the cell typing application further directs the processor to provide the 2D image via the display.

10. The cell typing system of claim 1, wherein the CNN further provides a confidence metric reflecting probability of the cell classification being correct.

11. A method for cell typing, comprising:
obtaining single cell ribonucleic acid sequencing (scRNA-seq) data generated from a single cell;
generating a two-dimensional (2D) image comprising a grid of pixels, where each pixel describes a gene-gene interaction based upon the scRNA-seq data;
providing the 2D image to a convolutional neural network (CNN);
obtaining a cell classification of the single cell from the CNN; and
providing the cell classification via a display.

12. The method for cell typing of claim 1, generating the 2D image comprises:
generating a pairwise interaction strength matrix that maximizes entropy of the scRNA-seq data;
generating a distance matrix for the 2D image, where the interaction strength matrix and the distance matrix have the same dimensions;
optimizing a transport function to produce a transport matrix coupling the distance matrix and the interaction strength matrix; and
transposing the scRNA-seq data into the 2D image using the transport matrix.

13. The method for cell typing of claim 12, wherein generating the pairwise interaction strength matrix comprises maximizing system entropy using a multivariate Gaussian distribution.

14. The cell method for cell typing of claim 13, wherein the transport function is optimized using a Gromov-Wasserstein discrepancy.

15. The method for cell typing of claim 14, wherein a loss function of the Gromov-Wasserstein discrepancy uses Kullback-Leibler divergence.

16. The method for cell typing of claim 14, wherein the Gromov-Wasserstein discrepancy is calculated using a regularized approximation.

17. The method for cell typing of claim 11, wherein the CNN is trained using a training data set comprising 2D images comprising a grid of pixels, where each pixel describes a gene-gene interaction based upon a different scRNA-seq data, where each 2D image is labeled with a cell type from which the different scRNA-seq data was obtained.

18. The method for cell typing of claim 11, further comprising generating the scRNA-seq data from a cell sample using a sequencer.

19. The method for cell typing of claim 11, further comprising providing the 2D image via the display.

20. The method for cell typing of claim 11, wherein the CNN further provides a confidence metric reflecting probability of the cell classification being correct.
